(19) United States
(12) Patent Application Publication
(10) Pub. No.: US 2026/0273229 A1
(43) Pub. Date: Sep. 17, 2026

(54) TWIST-LOCK LINKAGE FOR INTRODUCER SHEATH

(71) Applicant: ABIOMED, Inc., Danvers, MA (US)

(72) Inventor: Joseph Mayo, Danvers, MA (US)

(73) Assignee: ABIOMED, Inc., Danvers, MA (US)

(21) Appl. No.: 19/565,822

(22) Filed: Mar. 13, 2026

Related U.S. Application Data
(60) Provisional application No. 63/771,475, filed on Mar. 13, 2025.

(51) Int. Cl. A61M 25/06; A61M 25/00; A61M 39/00; A61M 39/10

(52) U.S. Cl. CPC A61M 25/0662; A61M 25/005; A61M 25/0097; A61M 39/1011; A61M 2039/0009; A61M 2205/582

(57) ABSTRACT

Locking devices configured to be disposed intermediate between, e.g., a sheath and a sheath lock, are provided. The locking device may include an inner and outer component. The inner component may include a substantially cylindrical body having a middle shelf portion located between the first and second end of the body. An outward-facing surface of the body includes an outer circumferential channel extending at least partially around the body. The first end has tab(s) or thread(s) extending outward, and the second end has tab(s) or thread(s) extending radially inward. The outer component surrounds the inner component, and includes a protrusion or pin extending inward to extend into the outer circumferential channel. A central channel is cut through partially through the locking device, extending radially inward from an outer surface of the outer component and passing partially though the inner component.

### CROSS-REFERENCE TO RELATED APPLICATIONS

The present application claims priority to U.S. Provisional Patent Application No. 63/771,475, filed Mar. 13, 2025, the contents of which are incorporated by reference herein in its entirety.

### TECHNICAL FIELD

The present disclosure is drawn to a linkage between two medical devices, and specifically, to a twist-lock linkage between a hub of a first medical device (such as an introducer sheath) and, e.g., a body of a second medical device (such as a sheath lock).

### BACKGROUND

A medical device, such as an intracardiac heart pump assembly, can be introduced into a patient in various ways. In general, a heart pump can be introduced in the heart to pump blood from the heart into a vessel to support the function of the heart. When deployed in the heart, a heart pump assembly pulls blood from the left ventricle of the heart and expels blood into the aorta, or pulls blood from the inferior vena cava (IVC) bypasses the right atrium and right ventricle and expels blood into the pulmonary artery. Heart pump assemblies are introduced surgically or percutaneously during a cardiac procedure through the vascular system. In one common approach, pump assemblies are inserted by a catheterization procedure through the femoral artery using a sheath, such as an introducer sheath. The sheath can alternatively be inserted in other locations such as in the femoral vein or any path for delivery of a pump for supporting either the left or right side of the heart. In some instances, after the pump is inserted, the introducer may be removed (e.g., if the introducer is a “peel-away” introducer), and may be replaced with a lower-profile repositioning sheath.

A sheath lock may sometimes be used to couple to a hub of the sheath, and allow a medical device to be inserted through the sheath lock and sheath and advanced into a blood vessel. However, in some instances, there may be an undesirable space, in a radial direction, between the outer surface of a medical device and an inner surface of the sheath. Such additional space may allow blood to accumulate. Accumulation of blood gives rise to thrombosis.

### BRIEF SUMMARY

In various aspects, a locking device for coupling medical devices may be provided. The locking device may include an inner component and an outer component disposed around the inner component. In certain arrangements, the outer component may be concentric to the inner component.

The inner component may have an outer wall forming a substantially cylindrical body defining a first end and a second end (axially distant from the first end). The substantially cylindrical body may have an inward-facing surface and an outward-facing surface. The substantially cylindrical body may include a middle shelf portion intermediate the first end and second end that extends in a radially inward from the inward-facing surface. The outward-facing surface may include an outer circumferential channel extending at least partially around the outward-facing surface intermediate between the first end and the second end. The outward-facing surface may have at least one tab or thread at the first end extending radially outward. The inward-facing surface may have at least one tab or thread at the second end extending radially inward.

The outer component may have an outer body surrounding the outward-facing surface of the inner component. The outer component may include a protrusion or pin extending radially inward. The protrusion or pin may be configured to extend into the outer circumferential channel of the inner component. The outer component may be a single piece. The outer component may be two or more pieces coupled together.

A central channel may be cut through the outer body and the substantially cylindrical body, extending radially inward from an outer surface of the outer body and passing through a portion of the outer wall forming the substantially cylindrical body and a portion of the middle shelf portion, the central channel having a defined width. The middle shelf portion may include an elevated portion surrounding a portion of the central channel, the elevation portion being a distance>0 mm away from the central channel, and extending towards either the first or second end.

In some aspects, the locking device may be free of other components. In some aspects, the locking device may include, e.g., a c-ring extending partially around the outer component.

The second end may include at least one slot cut through the outer wall (e.g., a slot cut through in a radial direction).

The outer component may have an outer body defining at least one cutout slot on a bottom surface. The cutout slot may extend only partially through the outer component in a direction radially outward from an inner surface towards the outer surface.

The protrusion or pin may be a pin that extends radially outward through an outer portion of the outer body. The protrusion or pin may be a pin that is enclosed by the outer body (e.g., sandwiched between a top and bottom portion of the outer body).

The substantially cylindrical body may be a rigid body, and the outer body may be a flexible body. An outer surface of the outer body may be smooth. An outer surface of the outer body may have one or more protrusions or ridges extending outward.

The locking device may include a tubular member coupled to the device. The tubular member may have a sidewall defining a first end and a second end and a lumen extending therethrough. The first end of the tubular member may be coupled to the middle shelf portion such that a device (e.g., a medical device) that extends through the lumen and out the first end will pass perpendicularly through the central channel. An outer surface of the sidewall may be in contact with the elevated portion of the middle shelf portion.

In various aspects, a system may be provided. The system may include a sheath, such as an introducer sheath, a sheath lock, and a locking device as disclosed herein. The first end of the locking device may be coupled to the sheath lock, and the second end of the locking device may be coupled to a hub of the sheath. The sheath may be a coil-reinforced sheath. The system may include a medical device extending through the sheath lock, through the locking device, and through the sheath. The locking device may be configured to provide haptic feedback to a user when the sheath lock and the outer component of the locking device are rotated.

### BRIEF DESCRIPTION OF FIGURES

FIG. 1A is an illustration of an exploded view of a locking device.

FIG. 1B is an illustration showing a second end of the inner component of a locking device.

FIG. 1C is an illustration of a cross-sectional side view of a locking device.

FIG. 1D is an illustration of a cross-sectional view of an inner component.

FIG. 1E is an illustration showing an embodiment of the inner component.

FIG. 1F is an illustration of a top view of an inner component, showing a channel with a varying radius.

FIGS. 2 and 3 are exploded views of multi-part outer components.

FIG. 4 is an illustration of an outer component.

FIG. 5 is an illustration of a locking device with a c-ring.

FIG. 6 is an illustration of an inner component coupled to a tubular member.

FIG. 7 is an illustration of a system.

### DETAILED DESCRIPTION

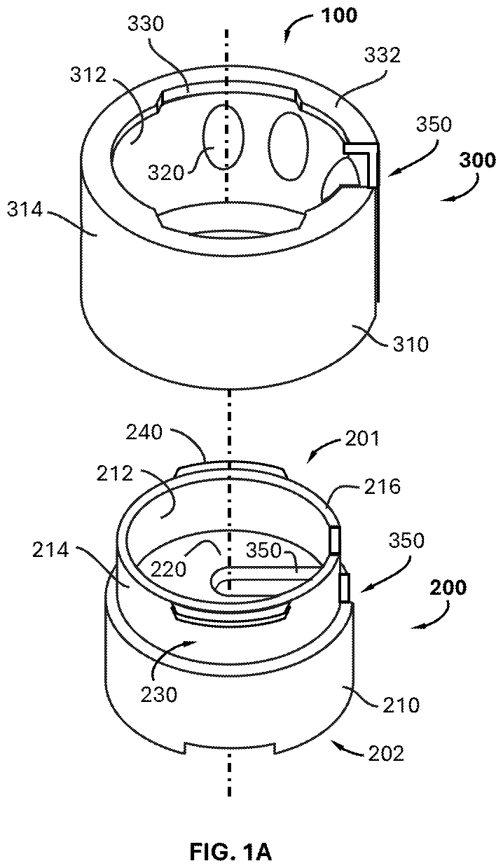

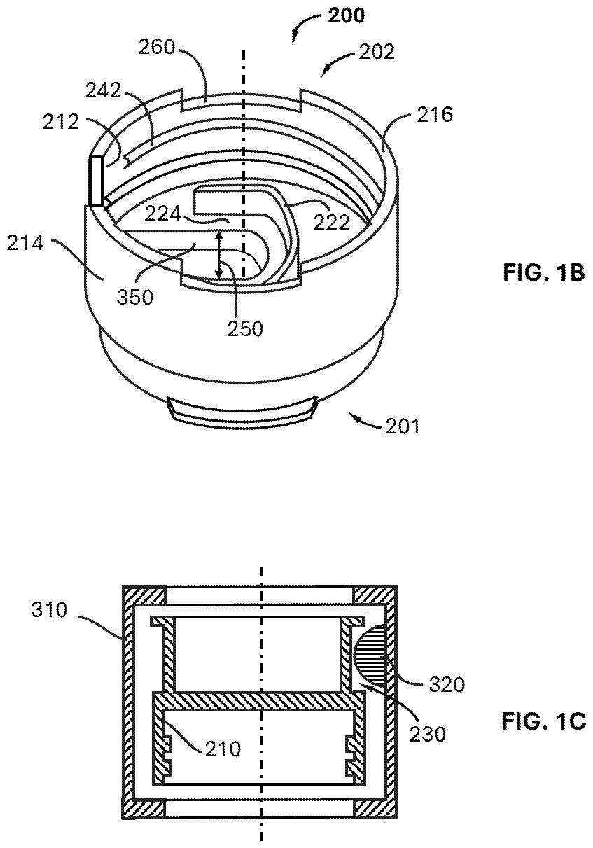

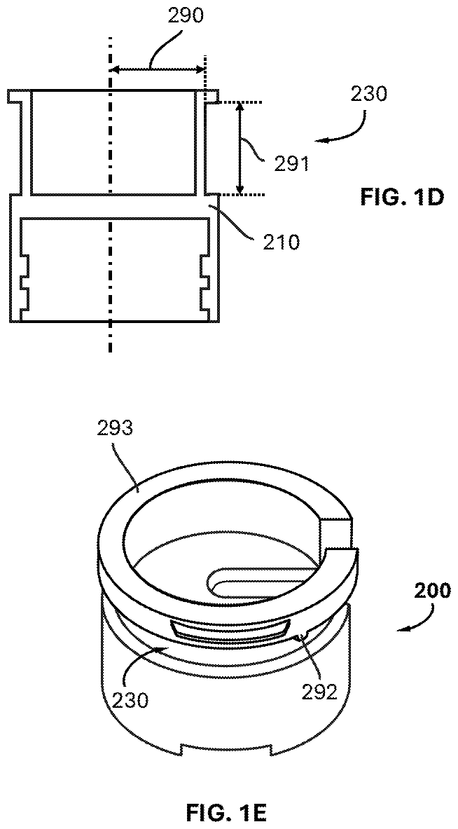

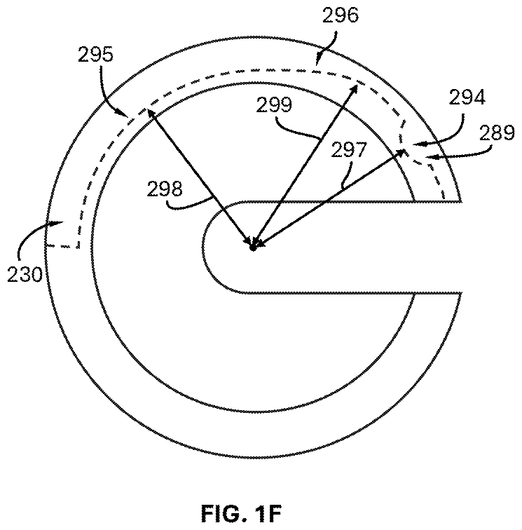

Locking devices for coupling medical devices may be provided. Referring to FIG. 1A, a locking device (100) may include an inner component (200) and an outer component (300). The outer component may be disposed around the inner component. In one embodiment, the outer component may be concentric to the inner component. The components may be made of any appropriate material, although in a preferred embodiment the two components each comprise polymeric materials, and in a more preferred embodiment, the two components each comprise different polymeric materials.

The inner component (200) may have an outer wall (216) forming a substantially cylindrical body (210) defining a first end (201) and a second end (202), where the second end is located an axially distance from the first end. The substantially cylindrical body may have an inward-facing surface (212) and an outward-facing surface (214).

The substantially cylindrical body may include a middle shelf portion (220) intermediate the first end (201) and second end (202) that extends in a radially inward from the inward-facing surface.

The outward-facing surface may include an outer circumferential channel (230) extending at least partially around the outward-facing surface intermediate between the first end and the second end. This outer circumferential channel may be considered a “track” within which a protrusion, pin, etc., may interface. Referring to FIG. 1D, in some embodiments, the outer circumferential channel (230) may have an outer radius (290). The outer radius may be constant. However, in some preferred embodiments, the outer radius may vary. The height of track (e.g., in the axial direction) may be constant, or may vary. For example, referring to FIG. 1E, the track may include a protrusion or bump (292) that extends in an axial direction from, e.g., a top edge (293) towards the opposite end of the inner component. As will be recognized, these height and radius of the track may be varied in order to provide various features.

The track may be configured to have, e.g., a “locked” and “unlocked” position. For example, referring to FIG. 1F, in a “locked” position (294), a radius of the channel may be a first radius (297). In an “unlocked” position (295), a radius of the channel may be a second radius (298). In some cases, the second radius (298) may be smaller than the first radius (297). In some cases, the second radius (298) may be the same as the first radius (297). In some cases, the second radius (298) may be larger than the first radius (297). There may be a recess (289) within the locked region. For example, in FIG. 1F, when locking the device by rotating the outer component, the change from a larger radius to the smaller radius in the recess may generate haptic feedback to indicate the correct position has been reached.

The track may be configured to have a “transition” position, between the locked and unlocked positions. For example, in the “transition” position (296), a radius may vary in the circumferential direction. The radius may vary in a continuous fashion, or there may be one or more step changes. The radius may include a third radius (199). The third radius may be, e.g., a maximum radius of the channel. There may be one or more recesses (where the radius decreases) or protrusion (where the radius increases) within the transition region, to provide haptic feedback. In some instances, the radius may generally increase across the circumferential length of the transition region.

The outward-facing surface (214) may have at least one tab or thread (240) at the first end (201) extending radially outward. The outer component (300) may have one or more slots (330) through an axial-facing surface (332) of the outer body (310), where each slot (330) generally aligns circumferentially with one of the tab(s) or thread(s) (240). Said differently, when the outer component is in place around the inner component, the slot (330) may allow the tab or thread (240) to be visible when viewed from above.

Referring briefly to FIG. 1B, the inward-facing surface (212) may have at least one tab or thread (242) at the second end (202) extending radially inward.

Referring again to FIG. 1A, the outer component (300) may have an outer body (310) configured to surround the outward-facing surface (214) of the inner component (200). The outer component (300) may include a protrusion or pin (320) extending radially inward.

Referring briefly to FIG. 1C, the protrusion or pin (320) may be configured to extend into the outer circumferential channel (230) of the substantially cylindrical body.

Referring to FIGS. 1A-1B, a central channel (350) may be cut through the outer body (310) and partially through the substantially cylindrical body (210), extending radially inward from an outer surface (314) of the outer body (310) and passing through a portion of the outer wall (216) forming the substantially cylindrical body (210) and a portion of the middle shelf portion (220).

The central channel extends the entire axial length of the locking device. The central channel may have a defined width (250). The defined width being perpendicular to the radially inward direction the channel is cut into the bodies. The width should be large enough to allow at least a portion of a medical device to pass through the channel. The width of the channel through the substantially cylindrical body of the inner component may be different from the width of the channel through the outer body.

The middle shelf portion (220) may include an elevated portion (222) surrounding a portion of the central channel (350), the elevation portion being a distance>0 mm away from the central channel, such that there may be a planar area (224) between the elevated portion (222) and the central channel (350). The elevated portion may extend towards either the first or second end.

The second end may include at least one slot (260) cut through the outer wall (216). The slot may extend fully or partially though the outer wall in a radial direction. The opening formed by the slot may be, e.g., surrounded on 3 or 4 sides by the outer wall (in FIG. 1B, it is shown as being surrounded on three sides, but it will be understood that a slot could be created that was surrounding on four sides). The opening may have any appropriate cross-sectional shape, such as that of an oval, a rectangle, pyramidal, etc.

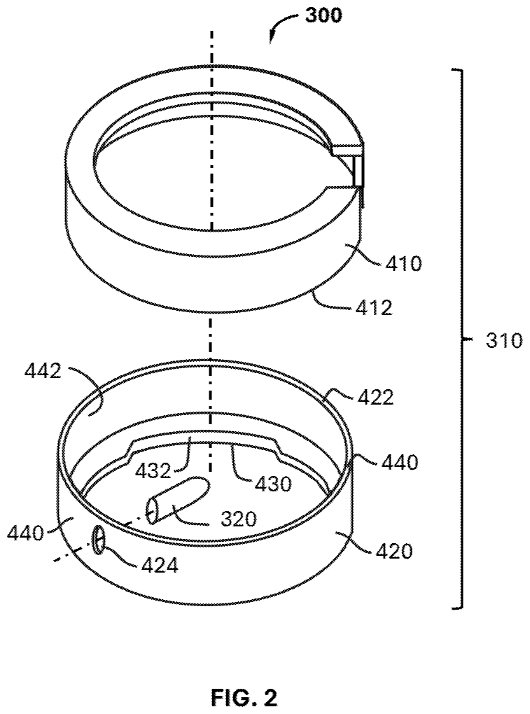

As shown in FIGS. 1A-1C, the outer component may be a single piece. However, the outer component may be two or more pieces coupled together. For example, referring to FIG. 2, the outer component may include a top body portion (410), a bottom body portion (420), and a separate pin (320). A bottom surface (412) of the top body portion may be coupled to a top surface (422) of the bottom body portion (420). This may involve, e.g., glue, welding, etc.

Whether one or two pieces are used, the outer component (300) may have an outer body (310) defining at least one cutout slot (432) on a bottom surface (430). The cutout slot may extend only partially through the outer component in a direction radially outward from an inner surface (442) towards the outer surface (444).

The protrusion or pin (320) may be a pin that extends radially outward through an outer portion of the outer body (e.g., through a sidewall (440) forming the outer body). For example, the pin may extend through an opening (424) defined by the sidewall (440).

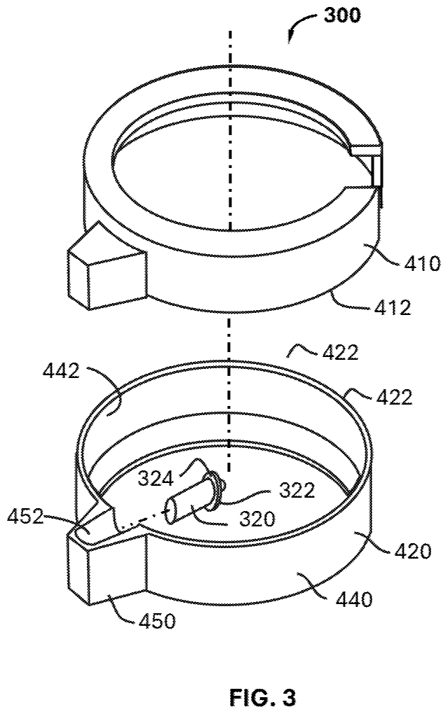

Referring to FIG. 3, the protrusion or pin (320) may be a pin that is enclosed by the outer body. As shown in FIG. 3, the pin may be disposed at least partially within a cavity (452) formed within a protruding portion (450) of the outer body. In some cases, when the top portion (410) and bottom portion (420) of the outer body are coupled together, the pin, within the cavity, may be sandwiched between the top and bottom portions. In some instances, the pin may include a stopping plate (322) that prevents the pin from sliding outward away beyond a certain point. For example, movement of the pin radially outward may be arrested when a face (324) of the stopping plate (322) contacts the inner surface (442) of the outer body.

While the relative stiffnesses of the inner and outer components may vary as appropriate, in a preferred embodiment, the substantially cylindrical body may be a rigid body, and the outer body may be a flexible body.

As used herein, the term “flexible” generally refers to the ability of an article to withstand stress without breakage or significant fracture when it is bent at, e.g., a 90° angle. Preferably, such article can undergo significant elastic deformation. Such articles may, in some instances, be characterized by a Young's Modulus of no more than 1 GPa, preferably no more than 0.5 GPa, more preferably no more than 0.25 GPa, most preferably no more than 0.2 GPa. As used herein, the term “rigid” generally refers to a non-flexible articles.

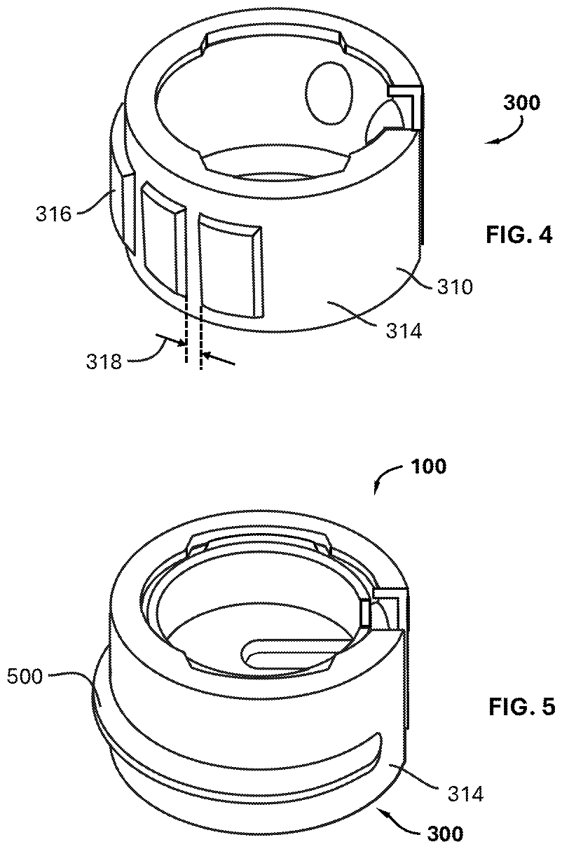

In some instances, the outer surface (314) of the outer body (310) may be smooth (see FIG. 1A). Referring to FIG. 4, in some instances, an outer surface (314) of the outer body (310) may have one or more protrusions or ridges (316) extending radially outward. Such may be useful for, e.g., providing grip for a user. The protrusions or ridges (316) may take any shape or form. The protrusions or ridges (316) may be symmetrically or asymmetrically arranged. If any space (318) between protrusions or ridges exist, such a space (318) between each protrusion or ridge may be constant, or may vary.

The locking device may be free of other components. However, in some instances, the locking device may include one or more other components.

Referring to FIG. 5, the locking device may include, e.g., a c-ring (500) extending partially around the outer component (300) (e.g., circumferentially, partially around outer surface (314)).

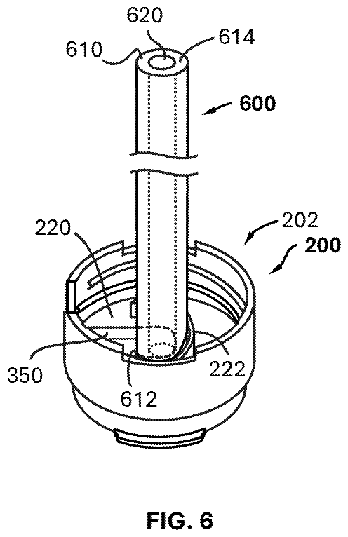

Referring to FIG. 6, the locking device may include a tubular member (600) coupled to the device. The tubular member (600) may have a sidewall (610) defining a first end (612) and a second end (614) and a lumen (620) extending therethrough. The first end of the tubular member may be coupled to the middle shelf portion (220) such that a device (e.g., a medical device) that extends through the lumen (620) and out the first end (612) will pass perpendicularly through the central channel (350) either immediately before entering the first end or immediately after exiting the first end, depending on the configuration of the locking device. An outer surface (616) of the sidewall (610) may be in contact with the elevated portion (222) of the middle shelf portion.

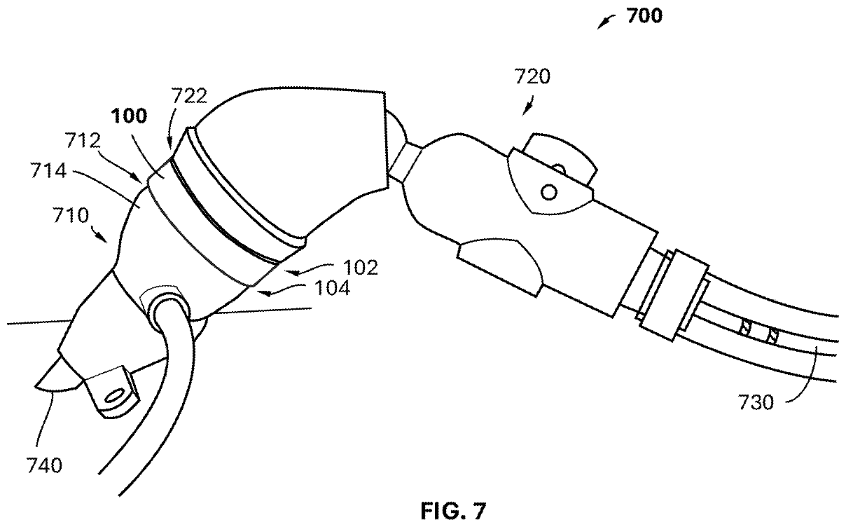

In various aspects, a system may be provided. Referring to FIG. 7, the system (700) may include a sheath (710), such as an introducer sheath, a sheath lock (720), and a locking device (100) as disclosed herein.

The first end (102) of the locking device (100) may be coupled to a proximal end (722) of the sheath lock (720). In some instances, the proximal end of the sheath lock (720) may be configured to couple to the at least one tab or thread (240) at the first end (201) of the substantially cylindrical body.

The second end (104) of the locking device (100) may be coupled to a distal end (712) of the sheath (710), and specifically to a hub (714) of the sheath. The sheath may be any appropriate sheath, such as a coil-reinforced sheath. Here, coil-reinforced generally indicates that the sidewalls forming the sheath may include one or more coils of a reinforcing material that is stiffer than the other materials forming the sidewall of the sheath. Such material may include, e.g., a metal wire such as a nitinol wire, a stainless-steel wire, etc., or a polymeric strand.

The protrusion or pin, in combination with the circumferential channel in which the protrusion or pin is disposed, may be configured to provide haptic feedback to a user when the sheath lock and the outer component of the locking device are rotated.

The system may include a medical device (730) extending through the sheath lock (720), through the locking device (100), and through the sheath (710). A portion of the sheath may be inserted into a patient via an access site (740), and the medical device may pass through the access site as well.

What is claimed is:

1. A locking device for coupling medical devices, comprising:
an inner component having an outer wall forming a substantially cylindrical body having an inward-facing surface and an outward-facing surface, the substantially cylindrical body defining a first end and a second end, the substantially cylindrical body including a middle shelf portion intermediate the first end and second end that extends in a radially inward from the inward-facing surface; where the outward-facing surface includes an outer circumferential channel extending at least partially around the outward-facing surface intermediate between the first end and the second end, where the outward-facing surface has at least one tab or thread at the first end extending radially outward, and where the inward-facing surface has at least one tab or thread at the second end extending radially inward; and
an outer component having an outer body surrounding the outward-facing surface, the outer component including a protrusion or pin extending radially inward, the protrusion or pin configured to extend into the outer circumferential channel of the inner component;
wherein a central channel is cut through the outer body and the substantially cylindrical body, extending radially inward from an outer surface and passing through a portion of the outer wall forming the substantially cylindrical body and a portion of the middle shelf portion, the central channel having a defined width; and
wherein the middle shelf portion includes an elevated portion surrounding a portion of the central channel, the elevated portion being a distance>0 mm away from the central channel, and extending towards either the first end or the second end.

2. The locking device of claim 1, wherein the outer component consists of a single piece.

3. The locking device of claim 2, wherein the locking device is free of other components.

4. The locking device of claim 2, further comprising a c-ring extending partially around the outer component.

5. The locking device of claim 1, wherein the outer component consists of two or more pieces coupled together.

6. The locking device of claim 1, wherein the second end includes at least one slot cut through the outer wall.

7. The locking device of claim 1, wherein the outer component has an outer body defining at least one cutout slot on a bottom surface, extending only partially through the outer component in a direction radially outward from an inner surface towards the outer surface.

8. The locking device of claim 1, wherein the protrusion or pin is a pin that extends radially outward through an outer portion of the outer body.

9. The locking device of claim 1, wherein the protrusion or pin is a pin that is enclosed by the outer body.

10. The locking device of claim 1, wherein the substantially cylindrical body is a rigid body, and the outer body is a flexible body.

11. The locking device of claim 1, wherein an outer surface of the outer body is smooth.

12. The locking device of claim 1, wherein an outer surface of the outer body has one or more protrusions or ridges extending outward.

13. The locking device of claim 1, further comprising a tubular member having a sidewall defining a first end and a second end and a lumen extending therethrough, the first end being coupled to the middle shelf portion such that a device extending through the lumen and out the first end will pass perpendicularly through the central channel, where an outer surface of the sidewall in contact with the elevated portion of the middle shelf portion.

14. A system, comprising:
a sheath;
a sheath lock; and
a locking device of claim 1, wherein the first end of the locking device is coupled to the sheath lock, and the second end of the locking device is coupled to a hub of the sheath.

15. The system of claim 14, further comprising a medical device extending through the sheath lock, through the locking device, and through the sheath.

16. The system of claim 14, wherein the sheath is an introducer sheath.

17. The system of claim 16, wherein the sheath is a coil-reinforced introducer sheath.

18. The system of claim 14, wherein the locking device is configured to provide haptic feedback to a user when sheath lock and the outer component are rotated.
